SEQUENCE LISTING (35 sequences)

SEQ ID NO: 1 - DNA, 45 bp, Artificial Sequence
  Chemically synthesized primer
cgtatcgcctccctcgcgccatcagggrattggaggaaatgaaca

SEQ ID NO: 2 - DNA, 47 bp, Artificial Sequence
  Chemically synthesized primer
ctatgcgccttgccagcccgctcagtgaaattrctgccattgtctgt

SEQ ID NO: 3 - DNA, 47 bp, Artificial Sequence
  Chemically synthesized primer
cgtatcgcctccctcgcgccatcagttggaggaaatgaacaagtaga

SEQ ID NO: 4 - DNA, 46 bp, Artificial Sequence
  Chemically synthesized primer
ctatgcgccttgccagcccgctcagtkactggccatcttcctgcta

SEQ ID NO: 5 - DNA, 46 bp, Artificial Sequence
  Chemically synthesized primer
cgtatcgcctccctcgcgccatcagtaaaattagcaggaagrtggc

SEQ ID NO: 6 - DNA, 47 bp, Artificial Sequence
  Chemically synthesized primer
ctatgcgccttgccagcccgctcagctgtctctgtaataaacccgaa

SEQ ID NO: 7 - DNA, 45 bp, Artificial Sequence
  Chemically synthesized primer
cgtatcgcctccctcgcgccatcaggtyaargcagcctgttggtg

SEQ ID NO: 8 - DNA, 48 bp, Artificial Sequence
  Chemically synthesized primer
ctatgcgccttgccagcccgctcagacaatcagcacctgccatctgtt

SEQ ID NO: 9 - DNA, 46 bp, Artificial Sequence
  Chemically synthesized primer
cgtatcgcctccctcgcgccatcagcaaatggcagtattcatycac

SEQ ID NO: 10 - DNA, 48 bp, Artificial Sequence
  Chemically synthesized primer
ctatgcgccttgccagcccgctcaggtgctttactaaactdttccatg

SEQ ID NO: 11 - DNA, 48 bp, Artificial Sequence
  Chemically synthesized primer
cgtatcgcctccctcgcgccatcagcaaagtcagggagtagtagartc

SEQ ID NO: 12 - DNA, 46 bp, Artificial Sequence
  Chemically synthesized primer
ctatgcgccttgccagcccgctcagtgttctaatcctcatcctgtc

SEQ ID NO: 13 - DNA, 10 bp, Artificial Sequence
  Chemically synthesized primer
cgtctagtac

SEQ ID NO: 14 - DNA, 10 bp, Artificial Sequence
  Chemically synthesized primer
tctacgtagc

SEQ ID NO: 15 - DNA, 10 bp, Artificial Sequence
  Chemically synthesized primer
tgtactactc

SEQ ID NO: 16 - DNA, 10 bp, Artificial Sequence
  Chemically synthesized primer
acgactacag

SEQ ID NO: 17 - DNA, 10 bp, Artificial Sequence
  Chemically synthesized primer
cgtagactag

SEQ ID NO: 18 - DNA, 10 bp, Artificial Sequence
  Chemically synthesized primer
tacgagtatg

SEQ ID NO: 19 - DNA, 10 bp, Artificial Sequence
  Chemically synthesized primer
tactctcgtg

SEQ ID NO: 20 - DNA, 10 bp, Artificial Sequence
  Chemically synthesized primer
tagagacgag

SEQ ID NO: 21 - DNA, 10 bp, Artificial Sequence
  Chemically synthesized primer
tcgtcgctcg

SEQ ID NO: 22 - DNA, 10 bp, Artificial Sequence
  Chemically synthesized primer
acatacgcgt

SEQ ID NO: 23 - DNA, 10 bp, Artificial Sequence
  Chemically synthesized primer
acgcgagtat

SEQ ID NO: 24 - DNA, 10 bp, Artificial Sequence
  Chemically synthesized primer
actactatgt

SEQ ID NO: 25 - DNA, 10 bp, Artificial Sequence
  Chemically synthesized primer
actgtacagt

SEQ ID NO: 26 - DNA, 10 bp, Artificial Sequence
  Chemically synthesized primer
agactatact

SEQ ID NO: 27 - DNA, 45 bp, Artificial Sequence
  Chemically synthesized primer
cgtatcgcctccctcgcgccatcagaaaggrattggaggaaatga

SEQ ID NO: 28 - DNA, 44 bp, Artificial Sequence
  Chemically synthesized primer
ctatgcgccttgccagcccgctcagtggctacatgractgctac

SEQ ID NO: 29 - DNA, 43 bp, Artificial Sequence
  Chemically synthesized primer
cgtatcgcctccctcgcgccatcagaattggagagcaatggct

SEQ ID NO: 30 - DNA, 44 bp, Artificial Sequence
  Chemically synthesized primer
ctatgcgccttgccagcccgctcagctgccattgtctgtrtgta

SEQ ID NO: 31 - DNA, 44 bp, Artificial Sequence
  Chemically synthesized primer
cgtatcgcctccctcgcgccatcagtagcaggaagatggccagt

SEQ ID NO: 32 - DNA, 44 bp, Artificial Sequence
  Chemically synthesized primer
ctatgcgccttgccagcccgctcagctgcactgtaycccccaat

SEQ ID NO: 33 - DNA, 46 bp, Artificial Sequence; identical to SEQ ID NO: 9
  Chemically synthesized primer

SEQ ID NO: 34 - DNA, 48 bp, Artificial Sequence; identical to SEQ ID NO: 10
  Chemically synthesized primer

SEQ ID NO: 35 - DNA, 23 bp, Artificial Sequence
  Chemically synthesized primer
gtgctttactaaactdttccatg

(19) United States
(12) Patent Application Publication
(10) Pub. No.: US 2012/0244523 A1
(43) Pub. Date: Sep. 27, 2012

(54) System and Method for Detection of HIV Integrase Variants

(75) Inventors: Elizabeth P. St. John, Guilford, CT (US); Birgitte B. Simen, Orange, CT (US)

(73) Assignee: 454 Life Sciences Corporation

(21) Appl. No.: 13/423,786

(22) Filed: Mar. 19, 2012

Related U.S. Application Data
(60) Provisional application No. 61/467,581, filed on Mar. 25, 2011.

(51) Int. Cl. C12Q 1/70

(52) U.S. Cl. 435/5

(57) ABSTRACT

An embodiment of a method for detecting low frequency occurrence of one or more HIV sequence variants associated with integrase is described that comprises the steps of: (a) generating a cDNA species from a plurality of RNA molecules in an HIV sample population; (b) amplifying a plurality of first amplicons from the cDNA species, wherein each first amplicon is amplified with a pair of nucleic acid primers capable of amplifying products from clades A, B, C, D, AE and G sub-types; (c) clonally amplifying the amplified copies of the first amplicons to produce a plurality of second amplicons; (d) determining a nucleic acid sequence composition of the second amplicons; (e) detecting one or more sequence variants that occur at a frequency of 5% or less in the nucleic acid sequence composition of the second amplicons; and (f) correlating the detected sequence variants with variation associated with HIV integrase.

### CROSS-REFERENCE TO RELATED APPLICATIONS

This application is related to and claims priority from U.S. Provisional Patent Application Ser. No. 61/467,581, titled “System and Method for Detection of HIV Integrase Variants”, filed Mar. 25, 2011. This application is also related to U.S. patent application Ser. No. 12/592,243, titled “System and Method for Detection of HIV Integrase Variants”, filed Nov. 19, 2009, each of which is hereby incorporated by reference herein in its entirety for all purposes.

### INCORPORATION-BY-REFERENCE OF SEQUENCE LISTING

The contents of the text file named “21465-541002US_ST25.txt,” which was created on Mar. 15, 2012 and is 8 KB in size, are hereby incorporated by reference in their entirety.

### FIELD OF THE INVENTION

The invention provides methods, reagents and systems for detecting and analyzing sequence variants associated with HIV-1 for HIV clades A, B, C, D, AE and G sub-types. The variants may include single nucleotide polymorphisms (SNPs), insertion/deletion variant (referred to as “indels”) and allelic frequencies, in a population of target polynucleotides in parallel. The invention also relates to a method of investigating by massively parallel sequencing of nucleic acids replicated by polymerase chain reaction (PCR), for the identification of mutations and polymorphisms of both known and unknown sequences. The invention involves using nucleic acid primers specifically designed to amplify a particular region and/or a series of overlapping regions of HIV RNA or its complementary DNA associated with a particular HIV characteristic or function such as the integrase region associated with HIV's ability to integrate the viral DNA into the cellular DNA. Also, the target sites for the primers have a low rate of mutation enabling consistent amplification of the nucleic acids in a target HIV nucleic acid population which are suspected of containing variants (also referred to as quasispecies) to generate individual amplicons. Thousands of individual HIV amplicons are sequenced in a massively parallel, efficient, and cost effective manner to generate a distribution of the sequence variants found in the populations of amplicons that enables greater sensitivity of detection over previously employed methods.

### BACKGROUND OF THE INVENTION

The Human Immunodeficiency Virus (generally referred to as HIV) continues to be a major problem worldwide, even though a plethora of compounds have been approved for treatment. Due to the error-prone nature of viral reverse transcriptase and the high viral turnover (t½=1-3 days), the HIV genome mutates very rapidly. For example, reverse transcriptase is estimated to generate, on average, one mutation per replication of the 9.7 Kb genome that does not dramatically affect the ability of the virus to propagate. This leads to the formation of ‘quasispecies’, where many different mutants exist in a dynamic relationship.

Those of ordinary skill in the art appreciate that HIV, distributed globally, is not a homogeneous virus. Throughout the world there are varying clades and subtypes of HIV that affect persons of every socioeconomic status. The greatest prevalence of the virus is currently seen in the Southern Africa. The 9 countries with the highest prevalence worldwide are all located in this sub region and are all affected at rates between 10-26% (UNAIDS 2009). Given this sequence variation and the fact that the clade of a specific sample is usually unknown prior to sequencing, it is important to target as many of the existing clades as possible.

HIV virus particles enter cells via the CD4 receptor and a co-receptor molecule, where after entry HIV integrase performs functions for integration of the HIV pro-virus into the cellular machinery as described by Lataillade and Kozal (Lataillade M and Kozal M J, The hunt for HIV-1 integrase inhibitors, AIDS Patient Care and STDs (2006) 20:489, which is hereby incorporated by reference herein in its entirety for all purposes) and includes the steps of (1) assembling a stable complex between the integrase protein and specific DNA sequences at the ends of the viral genome; (2) 3′ processing of the viral genome; (3) strand transfer; and (4) DNA gap repair and ligation.

The HIV integrase gene coding sequence is located close to the 3′ end of the Pol region, flanked in the genome by the reverse transcriptase RNase and Vif—the latter has a partially overlapping reading frame that begins at the 3′ end of the integrase. The integrase protein is encoded by 288 amiNO: acids (32 kDa) and is released from the Pol polyprotein by the viral protease. It is composed of three domains: an N-terminal domain containing a zinc finger motif, a C-terminal domain, and catalytic core domain in between. The core contains a DDE motif that is necessary for enzymatic function (Freed EO, HIV-1 replication, Somat Cell and Mol Genet (2001) 26:13, which is hereby incorporated by reference herein in its entirety for all purposes).

The FDA has approved the use of an integrase inhibitor commercially known as Isentress (Raltegravir) available from Merck & Co after efficacy was shown in clinical trials (Grinsztejn et al., Protocol 005 Team. Safety and efficacy of the HIV-1 integrase inhibitor raltegravir (MK-0518) in treatment-experienced patients with multidrug-resistant virus: a phase II randomised controlled trial. Lancet (2007) 369:1261; and Steigbigel et al., Raltegravir with optimized background therapy for resistant HIV-1 infection. N Engl J Med (2008). 359:339, each of which is hereby incorporated by reference herein in its entirety for all purposes). Raltegravir targets the third step in viral genome integration, strand transfer, and several mutations have been described that decrease sensitivity to this drug (Lataillade and Kozal incorporated by reference above; Van Laethem et al., A genotypic assay for the amplification and sequencing of integrase from diverse HIV-1 group M subtypes. J Virol Methods (2008) 153:176; and Paar et al., Genotypic antiretroviral resistance testing for human immunodeficiency virus type 1 integrase inhibitors on the TruGene™ sequencing system, J Clin Microbiol. 2008 December; 46(12):4087-90. Epub 2008 Oct. 22, each of which is hereby incorporated by reference herein in its entirety for all purposes). In addition to Raltegravir, numerous integrase inhibitors are in the pipeline at major pharmaceutical companies (Lataillade and Kozal incorporated by reference above). It is of great interest to be able to detect resistance-linked mutations in order to predict responses to HIV integrase inhibitors, in a manner analogous to the genotyping for resistance-linked mutations in the protease and reverse transcriptase genes (Kuritzkes D R et al., Performance characteristics of the TRUGENE HIV-1 genotyping kit and the Opengene DNA sequencing system, J Clin Microbiol (2003) 41:1594, which is hereby incorporated by reference herein in its entirety for all purposes).

Current HIV drug resistance assays are typically performed as population assays (Kuritzkes D R et al., Van Laethem et al., Paar et al., each incorporated by reference above), which are, by their nature, less sensitive than assays based on clonal separation of each viral strain. However, previously employed clonal analysis assays are extremely labor intensive and require separately testing thousands of cellular clones from each subject in order to achieve high sensitivity.

Long read-length 454 sequencing is ideally suited to generating thousands of clonal reads from multiple subjects in a single sequencing run. Therefore, efficient detection of these mutations through a sequence-based HIV integrase inhibitor resistance determination assay wherein clonal sequences are obtained directly from viral RNA quasispecies without a labor intensive cloning step is highly desirable and enables substantial advancement in knowledge of the disease and treatment possibilities from early detection. Further, embodiments of high throughput sequencing techniques enabled for what may be referred to as “Massively Parallel” processing have substantially more powerful analysis, sensitivity, and throughput characteristics than previous sequencing techniques. For example, the high throughput sequencing technologies employing HIV specific primers of the presently described invention are capable of achieving a sensitivity of detection of low abundance alleles that include a frequency of 1% or less of the allelic variants in a population. As described above, this is important in the context of detecting HIV variants, particularly for integrase variants where high sensitivity provides an important early detection mechanism that result in a substantial therapeutic benefit.

### SUMMARY OF THE INVENTION

Embodiments of the invention relate to the determination of the sequence of nucleic acids. More particularly, embodiments of the invention relate to methods and systems for detecting sequence variants using high throughput sequencing technologies.

An embodiment of a method for detecting low frequency occurrence of one or more HIV sequence variants associated with integrase is described that comprises the steps of: (a) generating a cDNA species from a plurality of RNA molecules in an HIV sample population; (b) amplifying a plurality of first amplicons from the cDNA species, wherein each first amplicon is amplified with a pair of nucleic acid primers capable of amplifying products from clades A, B, C, D, AE and G sub-types; (c) clonally amplifying the amplified copies of the first amplicons to produce a plurality of second amplicons; (d) determining a nucleic acid sequence composition of the second amplicons; (e) detecting one or more sequence variants that occur at a frequency of 5% or less in the nucleic acid sequence composition of the second amplicons; and (f) correlating the detected sequence variants with variation associated with HIV integrase.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument under computer control and a reaction substrate;

FIG. 2 is a simplified graphical example of the HIV viral genome representing the positional relationship of the protease/reverse transcriptase, integrase, and V3 regions;

FIGS. 3A and 3B are simplified graphical examples of embodiments of the positional relationship of amplicons relative to the HIV integrase region; and

FIG. 4 is a functional block diagram of one embodiment of a method for identifying variation associated with HIV integrase.

### DETAILED DESCRIPTION OF THE INVENTION

As will be described in greater detail below, embodiments of the presently described invention include systems and methods for designing target specific sequences or primer species specific to HIV variants, and using those primers for highly sensitive detection of sequence variants.

### a. General

The term “flowgram” generally refers to a graphical representation of sequence data generated by SBS methods, particularly pyrophosphate based sequencing methods (also referred to as “pyrosequencing”) and may be referred to more specifically as a “pyrogram”.

The term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a single cycle that is typically part of an iterative process of introduction of fluid solution to a reaction environment comprising a template nucleic acid molecule, where the solution may include a nucleotide species for addition to a nascent molecule or other reagent, such as buffers, wash solutions, or enzymes that may be employed in a sequencing process or to reduce carryover or noise effects from previous flows of nucleotide species.

The term “flow cycle” as used herein generally refers to a sequential series of flows where a fluid comprising a nucleotide species is flowed once during the cycle (i.e. a flow cycle may include a sequential addition in the order of T, A, C, G nucleotide species, although other sequence combinations are also considered part of the definition). Typically, the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “signal droop” as used herein generally refers to a decline in detected signal intensity as read length increases.

The term “test fragment” or “TF” as used herein generally refers to a nucleic acid element of known sequence composition that may be employed for quality control, calibration, or other related purposes.

The term “primer” as used herein generally refers to an oligonucleotide that acts as a point of initiation of DNA synthesis under conditions in which synthesis of a primer extension product complementary to a nucleic acid strand is induced in an appropriate buffer at a suitable temperature. A primer is preferably a single stranded oligodeoxyribonucleotide.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule. “Natural” nucleotide species include, e.g., adenine, guanine, cytosine, uracil, and thymine. Modified versions of the above natural nucleotide species include, without limitation, hypoxanthine, xanthine, 7-methylguanine, 5,6-dihydrouracil, and 5-methylcytosine.

The term “monomer repeat” or “homopolymers” as used herein generally refers to two or more sequence positions comprising the same nucleotide species (i.e. a repeated nucleotide species).

The term “homogeneous extension” as used herein generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products, such as those produced from Polymerase Chain Reaction or Ligase Chain Reaction techniques.

The term “variant” or “allele” as used herein generally refers to one of a plurality of species each encoding a similar sequence composition, but with a degree of distinction from each other. The distinction may include any type of variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations.

The term “allele frequency” or “allelic frequency” as used herein generally refers to the proportion of all variants in a population that is comprised of a particular variant.

The term “key sequence” or “key element” as used herein generally refers to a nucleic acid sequence element (typically of about 4 sequence positions, i.e., TGAC or other combination of nucleotide species) associated with a template nucleic acid molecule in a known location (i.e., typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (i.e., a “test fragment” or “TF” as referred to above) in a reaction well, where the accuracy of the sequence derived from TF sequence and/or Key sequence associated with the TF or in an adaptor associated with a target nucleic acid is compared to the known sequence composition of the TF and/or Key and used to measure of the accuracy of the sequencing and for quality control. In typical embodiments, a proportion of the total number of wells in a sequencing run will be keypass wells which may, in some embodiments, be regionally distributed.

The term “blunt end” as used herein is interpreted consistently with the understanding of one of ordinary skill in the related art, and generally refers to a linear double stranded nucleic acid molecule having an end that terminates with a pair of complementary nucleotide base species, where a pair of blunt ends are typically compatible for ligation to each other.

The term “sticky end” or “overhang” as used herein is interpreted consistently with the understanding of one of ordinary skill in the related art, and generally refers to a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”).

The term “SPR1” as used herein is interpreted consistently with the understanding of one of ordinary skill in the related art, and generally refers to the patented technology of “Solid Phase Reversible Immobilization” wherein target nucleic acids are selectively precipitated under specific buffer conditions in the presence of beads, where said beads are often carboxylated and paramagnetic. The precipitated target nucleic acids immobilize to said beads and remain bound until removed by an elution buffer according to the operator's needs (DeAngelis, Margaret M. et al: Solid-Phase Reversible Immobilization for the Isolation of PCR Products. Nucleic Acids Res (1995), Vol. 23:22; 4742-4743, which is hereby incorporated by reference herein in its entirety for all purposes).

The term “carboxylated” as used herein is interpreted consistently with the understanding of one of ordinary skill in the related art, and generally refers to the modification of a material, such as a microparticle, by the addition of at least one carboxyl group. A carboxyl group is either COOH or COO—.

The term “paramagnetic” as used herein is interpreted consistently with the understanding of one of ordinary skill in the related art, and generally refers to the characteristic of a material wherein said material's magnetism occurs only in the presence of an external, applied magnetic field and does not retain any of the magnetization once the external, applied magnetic field is removed.

The term “bead” or “bead substrate” as used herein generally refers to any type of solid phase particle of any convenient size, of irregular or regular shape and which is fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art.

The term “reaction environment” as used herein generally refers to a volume of space in which a reaction can take place typically where reactants are at least temporarily contained or confined allowing for detection of at least one reaction product. Examples of a reaction environment include but are not limited to cuvettes, tubes, bottles, as well as one or more depressions, wells, or chambers on a planar or non-planar substrate.

The term “virtual terminator” as used herein generally refers to terminators substantially slow reaction kinetics where additional steps may be employed to stop the reaction such as the removal of reactants.

Some exemplary embodiments of systems and methods associated with sample preparation and processing, generation of sequence data, and analysis of sequence data are generally described below, some or all of which are amenable for use with embodiments of the presently described invention. In particular, the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample should be prepared and processed from its raw form into template molecules amenable for high throughput sequencing. The processing methods may vary from application to application, resulting in template molecules comprising various characteristics. For example, in some embodiments of high throughput sequencing, it is preferable to generate template molecules with a sequence or read length that is at least comparable to the length that a particular sequencing method can accurately produce sequence data for. In the present example, the length may include a range of about 25-30 bases, about 50-100 bases, about 200-300 bases, about 350-500 bases, about 500-1000 bases, greater than 1000 bases, or any other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will, however, be appreciated that other methods of fragmentation, such as digestion using restriction endonucleases, may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements (i.e. such as Key elements or other type of quality control element), unique identifiers (also referred to as a multiplex identifier or “MID”) that encode various associations such as with a sample of origin or patient, or other functional element.

For example, some embodiments of the described invention comprise associating one or more embodiments of an MID element having a known and identifiable sequence composition with a sample, and coupling the embodiments of MID element with template nucleic acid molecules from the associated samples. The MID coupled template nucleic acid molecules from a number of different samples are pooled into a single “Multiplexed” sample or composition that can then be efficiently processed to produce sequence data for each MID coupled template nucleic acid molecule. The sequence data for each template nucleic acid is de-convoluted to identify the sequence composition of coupled MID elements and association with sample of origin identified. In the present example, a multiplexed composition may include representatives from about 384 samples, about 96 samples, about 50 samples, about 20 samples, about 16 samples, about 12 samples, about 10 samples, or other number of samples. Each sample may be associated with a different experimental condition, treatment, species, or individual in a research context. Similarly, each sample may be associated with a different tissue, cell, individual, condition, drug or other treatment in a diagnostic context. Those of ordinary skill in the related art will appreciate that the numbers of samples listed above are provided for exemplary purposes and thus should not be considered limiting.

In preferred embodiments, the sequence composition of each MID element is easily identifiable and resistant to introduced error from sequencing processes. Some embodiments of MID element comprise a unique sequence composition of nucleic acid species that has minimal sequence similarity to a naturally occurring sequence. Alternatively, embodiments of a MID element may include some degree of sequence similarity to naturally occurring sequence.

Also, in preferred embodiments, the position of each MID element is known relative to some feature of the template nucleic acid molecule and/or adaptor elements coupled to the template molecule. Having a known position of each MID is useful for finding the MID element in sequence data and interpretation of the MID sequence composition for possible errors and subsequent association with the sample of origin.

For example, some features useful as anchors for positional relationship to MID elements may include, but are not limited to, the length of the template molecule (i.e. the MID element is known to be so many sequence positions from the 5′ or 3′ end), recognizable sequence markers such as a Key element and/or one or more primer elements positioned adjacent to a MID element. In the present example, the Key and primer elements generally comprise a known sequence composition that typically does not vary from sample to sample in the multiplex composition and may be employed as positional references for searching for the MID element. An analysis algorithm implemented by application 135 may be executed on computer 130 to analyze generated sequence data for each MID coupled template to identify the more easily recognizable Key and/or primer elements, and extrapolate from those positions to identify a sequence region presumed to include the sequence of the MID element. Application 135 may then process the sequence composition of the presumed region and possibly some distance away in the flanking regions to positively identify the MID element and its sequence composition.

Some or all of the described functional elements may be combined into adaptor elements that are coupled to nucleotide sequences in certain processing steps. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In some embodiments, two sets of priming sequence regions (hereafter referred to as priming sequence A, and priming sequence B) may be employed for strand selection, where only single strands having one copy of priming sequence A and one copy of priming sequence B is selected and included as the prepared sample. In alternative embodiments, design characteristics of the adaptor elements eliminate the need for strand selection. The same priming sequence regions may be employed in methods for amplification and immobilization where, for instance, priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. patent application Ser. No. 12/156,242, titled “System and Method for Identification of Individual Samples from a Multiplex Mixture”, filed May 29, 2008; and U.S. patent application Ser. No. 12/380,139, titled “System and Method for Improved Processing of Nucleic Acids for Production of Sequencable Libraries”, filed Feb. 23, 2009, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid, such as a water based fluid suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, or fluorinated oils.

Further, some emulsion embodiments may employ surfactants that act to stabilize the emulsion, which may be particularly useful for specific processing methods such as PCR. Some embodiments of surfactant may include one or more of a silicone or fluorinated surfactant. For example, one or more non-ionic surfactants may be employed that include, but are not limited to, sorbitan monooleate (also referred to as Span™ 80), polyoxyethylenesorbitsan monooleate (also referred to as Tween™ 80), or in some preferred embodiments, dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments, a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template and amplified copies of the template, amplified copies complementary to the template, or combination thereof. Further, the solid substrate may be enabled for attachment of other type of nucleic acids, reagents, labels, or other molecules of interest.

After emulsion breaking and bead recovery, it may also be desirable in typical embodiments to “enrich” for beads having a successfully amplified population of substantially identical copies of a template nucleic acid molecule immobilized thereon. For example, a process for enriching for “DNA positive” beads may include hybridizing a primer species to a region on the free ends of the immobilized amplified copies, typically found in an adaptor sequence, extending the primer using a polymerase mediated extension reaction, and binding the primer to an enrichment substrate such as a magnetic or sepharose bead. A selective condition may be applied to the solution comprising the beads, such as a magnetic field or centrifugation, where the enrichment bead is responsive to the selective condition and is separated from the “DNA negative” beads (i.e. NO: or few immobilized copies).

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. Pat. Nos. 7,638,276; 7,622,280; 7,842,457; 7,927,797; and 8,012,690 and U.S. patent application Ser. No. 13/033,240, each of which is hereby incorporated by reference herein in its entirety for all purposes.

Also embodiments sometimes referred to as Ultra-Deep Sequencing, generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants comprising sequence composition associated with a research or diagnostic utility where the primers may be employed to amplify and provide insight into the distribution of sequence variants in the sample. For example, a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (first amplicons) is subsequently further amplified individually in separate reaction vessels such as an emulsion based vessel described above. The resulting amplicons (referred to herein as second amplicons), each derived from one member of the first population of amplicons, are sequenced and the collection of sequences are used to determine an allelic frequency of one or more variants present. Importantly, the method does not require previous knowledge of the variants present and can typically identify variants present at <1% frequency in the population of nucleic acid molecules.

Some advantages of the described target specific amplification and sequencing methods include a higher level of sensitivity than previously achieved and are particularly useful for strategies comprising mixed populations of template nucleic acid molecules. Further, embodiments that employ high throughput sequencing instrumentation, such as for instance embodiments that employ what is referred to as a PicoTiterPlate® array (also sometimes referred to as a PTP™ plate or array) of wells provided by 454 Life Sciences Corporation, the described methods can be employed to generate sequence composition for over 100,000, over 300,000, over 500,000, or over 1,000,000 nucleic acid regions per run or experiment and may depend, at least in part, on user preferences such as lane configurations enabled by the use of gaskets, etc. Also, the described methods provide a sensitivity of detection of low abundance alleles which may represent 1% or less of the allelic variants present in a sample. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; PCT Patent Application Serial No. US 2008/003424, titled “System and Method for Detection of HIV Drug Resistant Variants”, filed Mar. 14, 2008; and U.S. Pat. No. 7,888,034, titled “System and Method for Detection of HIV Tropism Variants”, filed Jun. 17, 2009; and U.S. patent application Ser. No. 12/592,243, titled “SYSTEM AND METHOD FOR DETECTION OF HIV INTEGRASE VARIANTS”, filed Nov. 19, 2009, each of which is hereby incorporated by reference herein in its entirety for all purposes.

Further, embodiments of sequencing may include Sanger type techniques, techniques generally referred to as Sequencing by Hybridization (SBH), Sequencing by Ligation (SBL), or Sequencing by Incorporation (SBI) techniques. The sequencing techniques may also include what are referred to as polony sequencing techniques; nanopore, waveguide and other single molecule detection techniques; or reversible terminator techniques. As described above, a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) using an enzymatic reaction process to produce light or via detection the release of H⁺ and measurement of pH change (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include, but are not limited to, mass tags and fluorescent or chemiluminescent labels. In typical embodiments, unincorporated nucleotides are removed, for example by washing. Further, in some embodiments, the unincorporated nucleotides may be subjected to enzymatic degradation such as, for instance, degradation using the apyrase or pyrophosphatase enzymes as described in U.S. patent application Ser. Nos. 12/215,455, titled “System and Method for Adaptive Reagent Control in Nucleic Acid Sequencing”, filed Jun. 27, 2008; and 12/322,284, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 500 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. Pat. No. 7,601,499, titled “Paired end sequencing”; and in U.S. patent application Ser. No. 12/322,119, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e., CCD camera) or confocal type architecture for optical detection, Ion-Sensitive Field Effect Transistor (also referred to as “ISFET”) or Chemical-Sensitive Field Effect Transistor (also referred to as “ChemFET”) for architectures for ion or chemical detection, a microfluidics chamber or flow cell, a reaction substrate, and/or a pump and flow valves. Taking the example of pyrophosphate-based sequencing, some embodiments of an apparatus may employ a chemiluminescent detection strategy that produces an inherently low level of background noise.

In some embodiments, the reaction substrate for sequencing may include a planar substrate, such as a slide type substrate, a semiconductor chip comprising well type structures with ISFET detection elements contained therein, or waveguide type reaction substrate that in some embodiments may comprise well type structures. Further, the reaction substrate may include what is referred to as a PTP™ array available from 454 Life Sciences Corporation, as described above, formed from a fiber optic faceplate that is acid-etched to yield hundreds of thousands or more of very small wells each enabled to hold a population of substantially identical template molecules (i.e., some preferred embodiments comprise about 3.3 million wells on a 70×75 mm PTP™ array at a 35 μm well to well pitch). In some embodiments, each population of substantially identical template molecule may be disposed upon a solid substrate, such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. Pat. No. 7,682,816, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. Nos. 7,323,305 and 7,575,865, both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. Pat. No. 7,927,797; and U.S. patent application Ser. No. 13/045,210, each of which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Celeron®, Core™, or Pentium® processor made by Intel Corporation, a SPARC® processor made by Sun Microsystems, an Athlon™, Sempron™, Phenom™, or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP, Windows Vista®, or Windows®_{—}7) from the Microsoft Corporation; the Mac OS X operating system from Apple Computer Corp. (such as Mac OS X v10.6 “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example, some commonly employed web browsers include Microsoft® Internet Explorer 8 available from Microsoft Corporation, Mozilla Firefox® 3.6 from the Mozilla Corporation, Safari 4 from Apple Computer Corp., Google Chrome from the Google™ Corporation, or other type of web browser currently known in the art or to be developed in the future. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

As described above, embodiments of the invention relate to systems and methods for detecting HIV integrase sequence variants in HIV clades A, B, C, D, AE and G sub-types from a sample, and in some embodiments the association of detected variants to resistance and/or sensitivity to drugs that target HIV integrase function. In some of the described embodiments identified variant sequence composition from a patient sample is associated with known integrase drug resistance and/or sensitivity types and the association information can be used to determine an appropriate therapeutic regimen. It will be appreciated by those of ordinary skill that the association may include a diagnostic correlation of detected variants with previously identified variation known to be associated with drug resistance and/or sensitivity, or as a newly discovered correlation of detected variants with a drug resistance and/or sensitivity phenotype of a sample. Other inventions that target alternative HIV regions such as the reverse transcriptase region and regions for determining tropism types are described in PCT Patent Application Serial No. US 2008/003424, titled “SYSTEM AND METHOD FOR DETECTION OF HIV DRUG RESISTANT VARIANTS”, filed Mar. 14, 2008; U.S. patent application Ser. No. 12/456,528, titled “SYSTEM AND METHOD FOR DETECTION OF HIV TROPISM VARIANTS”, filed Jun. 17, 2009; and U.S. patent application Ser. No. 12/592,243, titled “SYSTEM AND METHOD FOR DETECTION OF HIV INTEGRASE VARIANTS”, filed Nov. 19, 2009, each of which is incorporated by reference above.

Embodiments of the described invention typically include a two stage PCR technique (i.e. producing first and second amplicons as described above) using primer species targeted to amplify regions of HIV integrase known to be associated with drug resistance and/or sensitivity types, coupled with a sequencing technique that produces sequence information from thousands of viral particles in parallel which enables identification of the occurrence of HIV integrase types (based upon an association of the integrase types with the detected sequence composition of variants in the sample), even those types occurring at a low frequency in a sample. In fact, embodiments of the invention can detect integrase sequence variants present in a sample containing HIV viral particles in non-stoichiometric allele amounts, such as, for example, HIV integrase variants present at greater than 50%, less than 50%, less than 25%, less than 10%, less than 5% or less than 1%. The described embodiments enable such identification in a rapid, reliable, and cost effective manner.

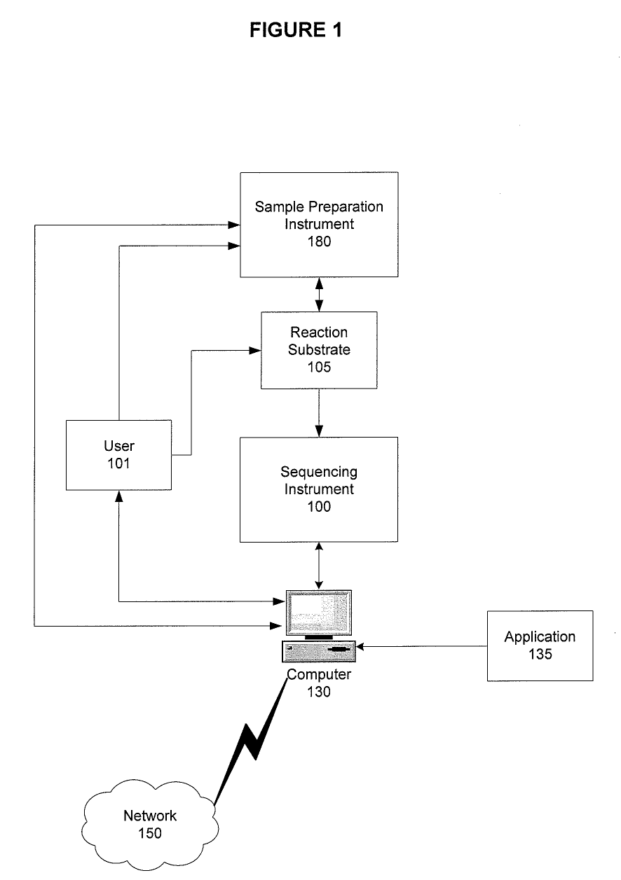

In a typical sequencing embodiment, one or more instrument elements may be employed that automate one or more process steps. For example, embodiments of a sequencing method may be executed using instrumentation to automate and carry out some or all process steps. FIG. 1 provides an illustrative example of sequencing instrument 100 that for sequencing processes requiring capture of optical signals typically comprise an optic subsystem and a fluidic subsystem for execution of sequencing reactions and data capture that occur on reaction substrate 105. It will, however, be appreciated that for sequencing processes requiring other modes of data capture (i.e. pH, temperature, electric current, electrochemical, etc.), a subsystem for the mode of data capture may be employed which are known to those of ordinary skill in the related art. For instance, a sample of template molecules may be loaded onto reaction substrate 105 by user 101 or some automated embodiment, then sequenced in a massively parallel manner using sequencing instrument 100 to produce sequence data representing the sequence composition of each template molecule. Importantly, user 101 may include any type of user of sequencing technologies.

In some embodiments, samples may be optionally prepared for sequencing in a fully automated or partially automated fashion using sample preparation instrument 180 configured to perform some or all of the necessary sample preparation steps for sequencing using instrument 100. Those of ordinary skill in the art will appreciate that sample preparation instrument 180 is provided for the purposes of illustration and may represent one or more instruments each designed to carry out some or all of the steps associated with sample preparation required for a particular sequencing assay. Examples of sample preparation instruments may include robotic platforms such as those available from Hamilton Robotics, Fluidigm Corporation, Beckman Coulter, or Caliper Life Sciences.

Further, as illustrated in FIG. 1, sequencing instrument 100 may be operatively linked to one or more external computer components, such as computer 130 that may, for instance, execute system software or firmware, such as application 135 that may provide instructional control of one or more of the instruments, such as sequencing instrument 100 or sample preparation instrument 180, and/or data analysis functions. Computer 130 may be additionally operatively connected to other computers or servers via network 150 that may enable remote operation of instrument systems and the export of large amounts of data to systems capable of storage and processing. In the present example, sequencing instrument 100 and/or computer 130 may include some or all of the components and characteristics of the embodiments generally described herein.

Typical design of primer target regions and sequence composition may be designed using alignments of known sequences using methods known to those of ordinary skill in the related art. For example, numerous sequence alignment methods, algorithms, and applications are available in the art including but not limited to the Smith-Waterman algorithm (Smith T F, Waterman M S (1981). “Identification of Common Molecular Subsequences”. Journal of Molecular Biology 147: 195-197, which is hereby incorporated by reference herein in its entirety for all purposes), BLAST algorithm (Altschul, S. F., Gish, W., Miller, W., Myers, E. W. & Lipman, D. J. (1990) “Basic local alignment search tool.” J. Mol. Biol. 215:403-410, which is hereby incorporated by reference herein in its entirety for all purposes), and Clustal (Thompson J D, Gibson T J, Plewniak F, Jeanmougin F, Higgins D G (1997). The ClustalX windows interface: flexible strategies for multiple sequence alignment aided by quality analysis tools. Nucleic Acids Research, 25:4876-4882. The alignment of sequences into a single sequence provides a consensus of the most frequent sequence composition of the population of HIV sequences.

Also in some embodiments, a software application may plot target regions for primer sequences against a representative or consensus sequence. Primer sets may then be designed to regions of the consensus sequence that are more conserved (i.e. less likely to mutate) than the regions of known mutation susceptibility having less conservation. Also, primer design includes additional considerations such as the length of the resulting amplification product with respect to the read length capabilities of the sequence technology employed to determine the sequence composition of the amplification products. The primer sets disclosed herein were designed to regions of a consensus sequence that are more conserved (i.e. less likely to mutate) than the regions of known mutation susceptibility. The advantage of targeting sequence regions with a low mutation rate for primer design includes the ability to reliably use the designed primers without substantial risk of failure due to variation at the target region that would render the primer unable to bind, as well as the possibility of using the same primer sets for multiple clades. In addition, those of ordinary skill in the art appreciate that certain positions within what may be considered “conserved” regions of the consensus sequence may still be variable in their composition and are considered “degenerate” positions. In some preferred embodiments, parameters used for primer design include inserting a degenerate base at a position in the primer composition in cases where there is less than 98% frequency of a nucleotide species at that position in a multiple sequence alignment used to determine the consensus sequence. In addition, other parameters that affect the selection of the binding target region and primer composition include restricting degenerate positions to those that have only two alternative nucleotide species, as well as restricting the primer composition to NO: more than two degenerate positions to reduce the risk of forming primer dimers in the amplification reaction. It is also desirable in some embodiments to restrict the presence of degenerate positions from the last 5 sequence positions of the primer composition (i.e. at the 3′ end of the forward primer and the 5′ end of the reverse primer) because it is advantageous to have the last 5 positions are highly conserved for binding efficiency. For example, a degenerate sequence position typically has multiple possible different nucleotide species that occur as alternative sequence composition at that position. Degenerate bases are well known in the art and various types of degeneracy are represented by IUPAC symbols that signify the alternative nucleotide compositions associated with the type. For example, the IUPAC symbol R represents that the purine bases (i.e. A and G) are alternative possibilities.

Those of ordinary skill in the art will appreciate that some variability of sequence composition for primer sets exist and that 90% or greater homology to the disclosed primer sequences are considered within the scope of the presently described invention. For example, the target regions for the sets of primers may be slightly shifted and thus some difference in primer sequence composition is expected. Also, refinements to the consensus sequence may be made or new sequence degeneracy at certain positions may be discovered resulting in a slight difference of sequence composition in the target region, and similarly some variation in primer sequence composition is expected.

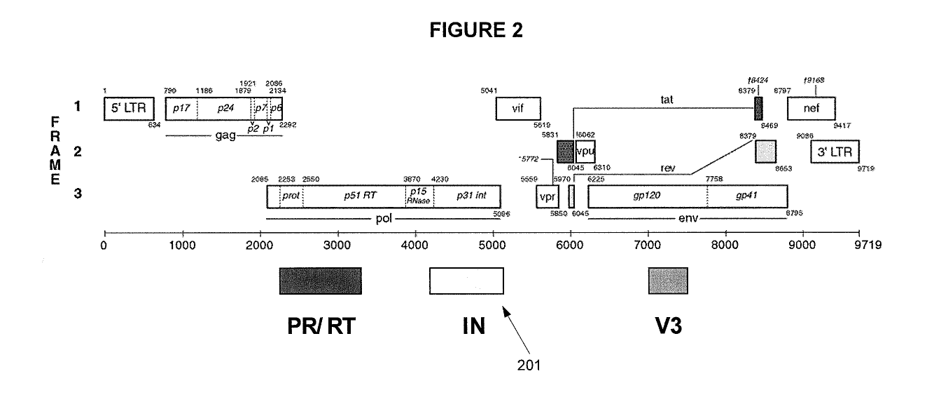

FIG. 2 provides an illustrative example of the HIV viral genome and the relative positions of the protease/reverse transcriptase, integrase, and V3 regions. More specifically the position of integrase region 201 that is flanked by the p15 and vif domains.

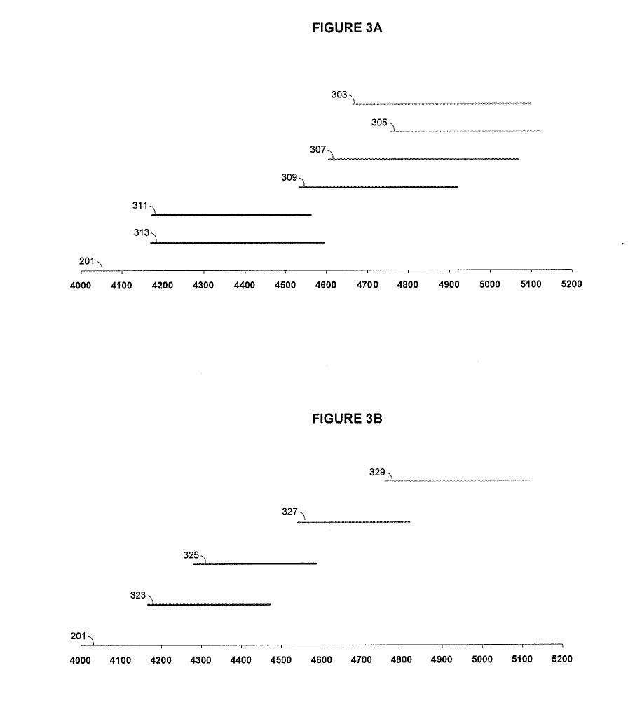

FIG. 3A provides an illustrative example of one embodiment comprising amplicons 303, 305, 307, 309, 311 and 313 arranged in a relationship that substantially provides at least double coverage (in some regions there is triple and quadruple coverage) of sequence composition of interest in integrase region 201. In some embodiments of the invention it is advantageous to produce amplicon products with overlapping coverage of the integrase region that can provide a substantial benefit in quality control as well as redundancy in the event that one of the amplicon products fails to amplify properly or suffers some other type of experimental artifact.

FIG. 3B provides an illustrative example of one embodiment comprising amplicons 323, 325, 327, and 329 arranged in a relationship that substantially provides a region of double coverage overlap between neighboring amplicons that span sequence composition of interest in integrase region 201. It will be appreciated that in some embodiments amplicons 305 and 329 may be substantially equivalent using the same or similar primer combinations to produce.

It will further be appreciated that exact relationship of illustrated amplicons in FIGS. 3A and 3B are provided for exemplary purposes should not be considered as limiting. It will also be appreciated that different amplicon products can be produced using different combinations of the primer sequences disclosed herein resulting in amplicons having different lengths and coverage than those illustrated in FIGS. 3A and 3B.

In typical embodiments, each amplicon is generated in a separate reaction using the associated primer combination for the desired amplicon. Further, in some embodiments the amplicons are longer than the length that can reliably be produced (i.e. with a low rate of amplification error, etc.) from amplification technologies such as PCR and thus each amplicon may be the result of 2 amplification products using the same primer combination. In the present example, the products typically will have a measure of overlap which again provides for assembly of the amplicon product and quality control. Tables 1 and 2 below provide an example of the relationship of the amplicons, amplicon length, and the primers used for their generation (see Examples for primer sequences).

TABLE 1

| amplicon | primer set |
|---|---|
| FIG. 3A | |
| Amplicon 303 (474bp) | Int 1F + Int 1R |
| Amplicon 305 (437bp) | Int 2F + Int 2R |
| Amplicon 307 (435bp) | Int 3F + Int 3R |
| Amplicon 309 (515bp) | Int 4F + Int 4R |
| Amplicon 311 (414bp) | Int 5F + Int 5R |
| Amplicon 313 (486bp) | Int 6F + Int 6R |
| FIG. 3B | |
| Amplicon 323 (375bp) | Int 1F* + Int 1R* |
| Amplicon 325 (375bp) | Int 2F* + Int 2R* |
| Amplicon 327 (349bp) | Int 3F* + Int 3R* |
| Amplicon 329 (434bp) | Int 4F* + Int 4R* |

In some embodiments, adaptor elements may be ligated to the ends of the amplicons during processing that comprise another general primer used for a second round of amplification from the individual amplicons producing a population of clonal copies (i.e. to generate second amplicons). It will be appreciated that the adaptors may also include other elements as described elsewhere in this specification such as quality control elements, other primers such as a sequencing primer and/or amplification primer (or single primer enabled to function as both an amplification and sequencing primer), unique identifier elements (i.e. MID elements as described above), and so on. Also, in some embodiments the target specific primers described above may be combined with one or more of the other elements useable in subsequent process steps. For example, a single stranded nucleic acid molecule may comprise the target specific primer sequence at one end with additional sequence elements adjacent. The target specific primer hybridizes to the target region may with the other elements hanging off due to the non-complementary nature of their sequence composition to the flanking sequence next to the target region, where the amplification product includes a copy of the region of interest as well as the additional sequence elements.

In some embodiments, a first strand cDNA is generated from HIV RNA using the target specific primers. In one embodiment, a first strand cDNA may be generated using a single primer that lacks a sequencing adaptor (also referred to as a SAD). Subsequently, the “first” amplicons are produced using the target specific primer/processing elements strategy. The resulting amplicons thus comprise the necessary processing elements due to their association with the primer.

Also in the described embodiments the second round of amplification typically occurs using the emulsion based PCR amplification strategy described above that results in an immobilized clonal population of “second” amplicons on a bead substrate that effectively sequesters the second amplicons preventing diffusion when the emulsion is broken. In the described embodiments, many of the second amplicons are then sequenced in parallel as described elsewhere in this specification (thousands, tens or hundreds of thousands, millions, etc., depending upon the limits of the sequencing technology). For example, beads with immobilized populations of second amplicons may be loaded onto reaction substrate 105 and processed using sequencing instrument 100 which generates >1000 clonal sequence reads from each sample and outputs the sequence data to computer 130 for processing. Computer 130 executes specialized software (such as for instance application 135) to identify variants that deviate from a consensus sequence that occur at 1% abundance or below from the sample.

In some embodiments the specialized software generates one or more consensus sequences using some or all of the sequence reads generated during the sequencing run, and thus the consensus sequence can be clade specific. For example, alignments and consensus sequences are generated from sequences produced from the sample of origin, which would be clade specific. In the presently described example, HXB2 (clade B) may be used as the general reference tool when making variant definitions within the AVA software, however the final variant determination is still taken from the clade/sample that is sequenced.

The sequence data may also be further analyzed by the same or different embodiment of software application to associate the sequence information from each read with known haplotypes associated with integrase type, where the sequence data from the individual reads may or may not include variation from the consensus sequence. The term “haplotype” as used herein generally refers to the combination of alleles associated with a nucleic acid sequence, which in the case of HIV includes the HIV RNA sequence. Those of ordinary skill in the art will appreciate that the association may include the use of one or more specialized data structures, such as for instance one or more databases, which store haplotype and/or integrase association information. The software application may include or communicate with the data structures in known ways to extract information from and/or provide new information into the data structure.

As described above, sequencing many nucleic acid templates in parallel provides the sensitivity necessary for the presently described invention. For example, based on binomial statistics the lower limit of detection (i.e., one event) for a fully loaded 60 mm×60 mm array of reaction wells (such as a PicoTiterPlate providing 2×10⁶ high quality bases, comprised of 200,000×100 base reads) with 95% confidence, is for a population with allelic frequency of at least 0.002%, and with 99% confidence for a population with allelic frequency of at least 0.003% 9 (it will also be appreciated that a 70×75 mm array of reaction wells could be employed as described above, which allows for an even greater number of reads and thus increased sensitivity). For comparison, SNP detection via pyrophosphate based sequencing has reported detection of separate allelic states on a tetraploid genome, so long as the least frequent allele is present in 10% or more of the population (Rickert et al., 2002 BioTechniques. 32:592-603). Conventional fluorescent DNA sequencing is even less sensitive, experiencing trouble resolving 50/50 (i.e., 50%) heterozygote alleles (Ahmadian et al., 2000 Anal. BioChem. 280:103-110).

Table 2 shows the probability of detecting zero, or one or more, events, based on the incidence of SNP's in the total population, for a given number N (=100) of sequenced amplicons. “*” indicates a probability of 3.7% of failing to detect at least one event when the incidence is 5.0%; similarly, “**” reveals a probability of 0.6% of failing to detect one or more events when the incidence is 7%.

The table thus indicates that the confidence level to detect a SNP present at the 5% level is 95% or better and, similarly, the confidence of detecting a SNP present at the 7% level is 99% or better.

TABLE 2

| | Prob. of at least 1 event | Prob. of NO: event |
|---|---|---|
| Incidence (%) | (N = 100) | (N = 100) |
| 1 | 0.264 | 0.736 |
| 2 | 0.597 | 0.403 |
| 3 | 0.805 | 0.195 |
| 4 | 0.913 | 0.087 |
| 5 | 0.963 | 0.037* |
| 6 | 0.985 | 0.015 |
| 7 | 0.994 | 0.006** |
| 8 | 0.998 | 0.002 |
| 9 | 0.999 | 0.001 |
| 10 | 1.000 | 0.000 |

Naturally, multiplex analysis is of greater applicability than depth of detection and Table 3 displays the number of SNPs that can be screened simultaneously on a single multi-reaction array, with the minimum allelic frequencies detectable at 95% and 99% confidence.

TABLE 3

| | | Minimum frequency | Minimum frequency |
|---|---|---|---|
| | | of SNP in population | of SNP in population |
| SNP | Number of | detectable with 95% | detectable with 99% |
| Classes | Reads | confidence | confidence |
| 1 | 200000 | 0.002% | 0.003% |
| 2 | 100000 | 0.005% | 0.007% |
| 5 | 40000 | 0.014% | 0.018% |
| 10 | 20000 | 0.028% | 0.037% |
| 50 | 4000 | 0.14% | 0.18% |
| 100 | 2000 | 0.28% | 0.37% |
| 200 | 1000 | 0.55% | 0.74% |
| 500 | 400 | 1.39% | 1.85% |
| 1000 | 200 | 2.76% | 3.64% |

### Examples

In the presently described embodiments, HIV integrase nucleotide sequences representing clades A, B, C, D, AE and clade G sub-types covering the regions of interest were obtained from the Los Alamos HIV Sequence Database and processed with the BioEdit Sequence Alignment Editor software. The software created a list of all nucleotide species identified at each sequence position in the alignment, which was exported to Microsoft Excel for calculation of the frequency of occurrence for each nucleotide species identified at each sequence position. The nucleotide species occurring at the highest frequency at each sequence position was designated as the nucleotide species represented in a consensus sequence for subsequent alignments. Further, a nucleotide species at a sequence position was designated as evolutionarily “conserved” if the consensus nucleotide species accounted for >98% frequency at that position.

Further analysis of the consensus sequence and nucleotide species frequency values at each sequence position revealed contiguous or semi-contiguous regions of sequence positions designated as conserved that were identified as candidate primer target regions. In particular, the five sequence positions at the 3′-most end of the candidate primer target regions were considered as important for efficient primer binding, and thus were more important to be listed as conserved. However, one of the sequence positions within the five sequence positions at the 3′-most end of the candidate primer target regions, although not the ultimate 3′ base, could consist of two distinct nucleotide species whose combined frequencies added up to >98% frequency, and were designated as a degenerate sequence position as indicated in the primer sequence design using one of the standard IUPAC-IUB degeneracy codes. In some embodiments of a primer sequence design one more degenerate position at another sequence position within the candidate primer sequence composition was also allowed. NO: more than one N or 3-base degeneracy (or, alternatively, two 2-base degeneracies (R, Y, K, M, S, W)) was allowed for a given primer.

As described above, the primer sequence designs for the Integrase region were derived from multi-sequence alignments for HIV-Clades A, B, C, D, AE and G sequences downloaded from the Los Alamos Database HIV Compendium. Shown below is a table of the number of sequences included in the multi-sequence alignments used to create a consensus for primer design.

TABLE 4

Clade

| | A | B | C | D | AE | G |
|---|---|---|---|---|---|---|
| Sequence | 67 | 1624 | 924 | 172 | 294 | 138 |
| number | | | | | | |

In addition to the consensus sequence obtained from the multi-sequence alignments obtained for the Integrase region, alignments were also made for the p15 and vif regions of the virus which flank the Integrase region in the viral genome. The sequences obtained for both p15 and the vif regions were compiled just as those for the Integrase region and then added to either end of the Integrase sequence so that primers could be designed beyond the boundaries of the Integrase region. In this way, the whole Integrase region sequence is completely covered by amplicons, without compromising coverage.

The number of multi-sequence alignments downloaded for the vif and p15 sequences are found in the table below:

TABLE 5

Clade

| | A | B | C | D | AE | G |
|---|---|---|---|---|---|---|
| Vif | 27 | 1460 | 587 | 105 | 178 | 37 |
| sequence | | | | | | |
| number | | | | | | |
| P15 | 12 | 1620 | 1073 | 1073 | 166 | 39 |
| sequence | | | | | | |
| number | | | | | | |

Not all of the sequences downloaded cover the region of interest for each amplicon. The table below indicates the coverage for each amplicon for each respective clade. Each cell shows the average number of sequences for the Forward (F) and Reverse (R) primer on a per-amplicon basis.

TABLE 6

| | Clade | | | | | |
|---|---|---|---|---|---|---|
| | A | B | C | D | AE | G |
| Amplicon | F: 12 | F: 1620 | F: 879 | F: 79 | F: 166 | F: 37 |
| Int1 | R: 67 | R: 1320 | R: 858 | R: 161 | R: 192 | R: 134 |
| Amplicon | F: 12 | F: 1620 | F: 753 | F: 79 | F: 166 | F: 37 |
| Int2 | R: 67 | R: 1402 | R: 852 | R: 165 | R: 205 | R: 133 |
| Amplicon | F: 21 | F: 1300 | F: 886 | F: 103 | F: 202 | F: 128 |
| Int3 | R: 19 | R: 1320 | R: 800 | R: 110 | R: 198 | R: 115 |
| Amplicon | F: 65 | F: 1404 | F: 864 | F: 158 | F: 205 | F: 134 |
| Int4 | R: 12 | R: 1310 | R: 790 | R: 160 | R: 198 | R: 115 |
| Amplicon | F: 67 | F: 1395 | F: 874 | F: 160 | F: 198 | F: 134 |
| Int5 | R: 27 | R: 1450 | R: 584 | R: 105 | R: 178 | R: 35 |
| Amplicon | F: 66 | F: 1400 | F: 857 | F: 163 | F: 197 | F: 133 |
| Int6 | R: 10 | R: 1320 | R: 792 | R: 95 | R: 95 | R: 95 |

Primer design was first performed using the Clade B consensus sequence generated from the multiple sequence alignments described above. Primers were then designed for clade B, targeted to those regions with regions conserved at greater than 98%. The newly designed primers were then aligned against a consensus sequence that had been generated for HIV-Clade C. To account for minor differences between clades, either degenerate primers were added to the sequences or the primers were shifted to a different location that would accommodate both clades. Once the combined clade B and C primer targets were identified they were then aligned against the Clade A consensus sequence. The same process was repeated for each of the clades for which primer designs were needed. Importantly, clades C, B, and A were selected as the first to find primer target regions due to their importance as the most commonly found clades.

In addition to targeting primers to regions of the virus that are highly conserved (>98%) and having the last 5 base pairs at the 3′ end highly conserved, additional constraints were also imposed when designing amplicons for the Integrase region: 1) that the amplicons were not to differ in length from each other by greater than 200 bp; 2) the amplicon primers would not cover any major, previously identified resistance mutations; 3) the primer designs would contain NO: more than two degenerate positions; 4) the G/C content of the primers would be as close to 50% as possible; and 5) that all regions of interest would be covered by overlapping amplicons.

The amplicon design shown below allows for dual read coverage at each nucleotide position of the Integrase region. The amplicon sizes allow for complete read through, of both the forward and reverse directions, using 454 Sequencing. The expected amplicon sizes, with adaptors and without MIDs (would add ˜20 base pairs in length) are as follows: Int 1=474, Int2=437, Int3=435, Int4=515, Int5=414, Int6=486.

Int 1F
(SEQ ID NO: 1)
CGTATCGCCTCCCTCGCGCCATCAGGGRATTGGAGGAAATGAACA
Int 1R
(SEQ ID NO: 2)
CTATGCGCCTTGCCAGCCCGCTCAGTGAAATTRCTGCCATTGTCTGT
Int 2F
(SEQ ID NO: 3)
CGTATCGCCTCCCTCGCGCCATCAGTTGGAGGAAATGAACAAGTAGA
Int 2R
(SEQ ID NO: 4)
CTATGCGCCTTGCCAGCCCGCTCAGTKACTGGCCATCTTCCTGCTA
Int 3F
(SEQ ID NO: 5)
CGTATCGCCTCCCTCGCGCCATCAGTAAAATTAGCAGGAAGRTGGC
Int 3R
(SEQ ID NO: 6)
CTATGCGCCTTGCCAGCCCGCTCAGCTGTCTCTGTAATAAACCCGAA
Int 4F
(SEQ ID NO: 7)
CGTATCGCCTCCCTCGCGCCATCAGGTYAARGCAGCCTGTTGGTG
Int 4R
(SEQ ID NO: 8)
CTATGCGCCTTGCCAGCCCGCTCAGACAATCAGCACCTGCCATCTGTT
Int 5F
(SEQ ID NO: 9)
CGTATCGCCTCCCTCGCGCCATCAGCAAATGGCAGTATTCATYCAC
Int 5R
(SEQ ID NO: 10)
CTATGCGCCTTGCCAGCCCGCTCAGGTGCTTTACTAAACTDTTCCATG
Int 6F
(SEQ ID NO: 11)
CGTATCGCCTCCCTCGCGCCATCAGCAAAGTCAGGGAGTAGTAGARTC
Int 6R
(SEQ ID NO: 12)
CTATGCGCCTTGCCAGCCCGCTCAGTGTTCTAATCCTCATCCTGTC

The 454 sequencing adaptors (GS FLX Titanium and GS Junior) are indicated as underlined sequence composition, whereas the un-underlined sequence composition is the target specific portion. MIDs (multiplex identifiers) for the Integrase amplicons are listed below. The MID sequence is inserted between the sequencing adaptor and the gene specific primer sequences. This sequence allows for identification of each sequence read for traceability back to the sample the read is derived from.

(SEQ ID NO: 13)
MID17 CGTCTAGTAC
(SEQ ID NO: 14)
MID18 TCTACGTAGC
(SEQ ID NO: 15)
MID19 TGTACTACTC
(SEQ ID NO: 16)
MID20 ACGACTACAG
(SEQ ID NO: 17)
MID21 CGTAGACTAG
(SEQ ID NO: 18)
MID22 TACGAGTATG
(SEQ ID NO: 19)
MID23 TACTCTCGTG
(SEQ ID NO: 20)
MID24 TAGAGACGAG
(SEQ ID NO: 21)
MID25 TCGTCGCTCG
(SEQ ID NO: 22)
MID26 ACATACGCGT
(SEQ ID NO: 23)
MID27 ACGCGAGTAT
(SEQ ID NO: 24)
MID28 ACTACTATGT
(SEQ ID NO: 25)
MID29 ACTGTACAGT
(SEQ ID NO: 26)
MID30 AGACTATACT

The primer design for single amplicon coverage of the Integrase region is shown in the figure below. The expected amplicon sizes, with adaptors and without MIDs are as follows: Int 1*=375 bp, Int 2*=375 bp, Int3*=349 bp Int 5=434 bp.

Int 1F*
(SEQ ID NO: 27)
CGTATCGCCTCCCTCGCGCCATCAGAAAGGRATTGGAGGAAATGA
Int 1R*
(SEQ ID NO: 28)
CTATGCGCCTTGCCAGCCCGCTCAGTGGCTACATGRACTGCTAC
Int 2F*
(SEQ ID NO: 29)
CGTATCGCCTCCCTCGCGCCATCAGAATTGGAGAGCAATGGCT
Int 2R*
(SEQ ID NO: 30)
CTATGCGCCTTGCCAGCCCGCTCAGCTGCCATTGTCTGTRTGTA
Int 3F*
(SEQ ID NO: 31)
CGTATCGCCTCCCTCGCGCCATCAGTAGCAGGAAGATGGCCAGT
Int 3R*
(SEQ ID NO: 32)
CTATGCGCCTTGCCAGCCCGCTCAGCTGCACTGTAYCCCCCAAT
Int 4F*
(SEQ ID NO: 33)
CGTATCGCCTCCCTCGCGCCATCAGCAAATGGCAGTATTCATYCAC
Int 4R*
(SEQ ID NO: 34)
CTATGCGCCTTGCCAGCCCGCTCAGGTGCTTTACTAAACTDTTCCATG

The 454 sequencing adaptors (GS FLX Titanium and GS Junior) are indicated as underlined sequence composition, whereas the un-underlined sequence composition is the target specific portion. MIDs (multiplex identifiers) for the Integrase amplicons are the same as those listed above.

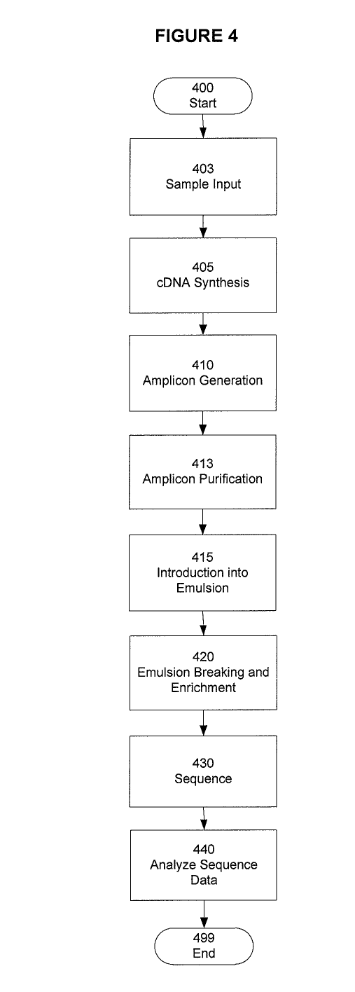

FIG. 4 provides an illustrative example of one embodiment of a method for identification of low frequency variation in the HIV integrase region that includes step 403 for initial sample input. In order to consistently detect minor variants down to 3% frequency, HIV-1 RNA samples used for in the method require a minimum viral content of 160 IU/μl as determined with an embodiment of a HIV real-time quantitative PCR assay. For detection down to 1% frequency, the minimum viral content should be at least 500 IU/μl. It will be appreciated by those of ordinary skill in the art that additional sources of systemic error may be introduced, such as for instance a low amount of error introduced from PCR processes, and the 1% refers to the frequency of variation and not systemic error.

If it is not practical to quantify the RNA samples, the RNA extraction can be performed on at least 140 μl of plasma into a total eluate of maximum 60 μl if the original viral load in the plasma is 100,000 copies per ml. For lower viral loads, scale the amount of plasma accordingly and pellet the virus for 1 hour 30 minutes at 20,600 rpm 4° C. Remove enough supernatant to leave 140 μl concentrate for the extraction procedure. Set up PCR and sequence duplicate reactions for several samples to verify consistent detection of low-frequency variants.

Next, the RNA sample is processed as illustrated in step 405 to generate a cDNA template from an HIV sample population. Generating the cDNA from the sample may be performed using the following procedure:
1. Place 96 well plate in cooler
2. Add 12.5 μl RNA per well
3. Add 0.5 μl primer Int 5R
   cDNA-Int 5R:

(SEQ ID NO: 35)
5′ GTGCTTTACTAAACTDTTCCATG 3′

Incubate at 65° C. for 10 minutes then place tube immediately on ice.
Prepare the Reverse Transcriptase (RT) mix scaled up for number of tubes:
1. Transcriptor RT reaction buffer (available from Roche) 4 μl
2. Protector RNase Inhibitor (available from Roche) 0.5 μl
3. dNTPs 2 μl
4. Transcriptor Reverse Transcriptase (available from Roche) 0.5 μl
   Mix briefly by vortexing and keep on ice until added to the RNA sample.
5. Add 8 μl RT mix per well
6. Seal plate and centrifuge briefly
7. Place in thermocycler and run the following cDNA program
   60 min, 50° C.
   5 min, 85° C.
   4° C. forever
8. Add 1 μl RNAse H (available from New England Biolabs) per well
9. Place in thermocycler block at 37° C. (with heated lid set at or above 50° C.) for 20 min.
10. Proceed immediately to amplicon generation or store the cDNA at −80° C.

Subsequently, as illustrated in step 410, pairs of region specific primers are employed to amplify target region from the cDNA templates generated in step 405 using the following procedure.
1. The 13× mix described below is sufficient for one 96 well plate (6 amplicons, 47 samples+1 control). The method can be scaled up or down as necessary.
2. Label 6 1.5 ml centrifuge tubes “IN1”, “IN2”, “IN3”, “IN4”, “IN5”, and “IN6”.

These labels refer to the following amplicons/primer sets:

| | |
|---|---|
| IN1 | Int 1F + Int 1R |
| IN2 | Int 2F + Int 2R |
| IN3 | Int 3F + Int 3R |
| IN4 | Int 4F + Int 4R |
| IN5 | Int 5F + Int 5R |
| IN6 | Int 6F + Int 6R |

3. If Multiplex Identifiers (MIDs) are required for the experiment, then for each set of amplicons add in the corresponding MID primer. E.g. if using MID1, then all primers of primer set IN1 should have MID1 added into the primer for both the forward and reverse directions. MID sequence is 10 base pairs long and should be inserted into the primer following the sequence adaptor sequence and immediately prior to the target primer sequence.
4. In each tube, prepare a PCR master mix with the primer set indicated by the label:

| | |
|---|---|
| 1x mix | 13x mix |

| | | | | |
|---|---|---|---|---|
| Forward primer | 1 | μl | 13 | μl |
| Reverse primer | 1 | μl | 13 | μl |
| dNTP mix | 0.5 | μl | 6.5 | μl |
| FastStart 10x buffer #2 | 2.5 | μl | 32.5 | μl |
| FastStart Hifi polymerase | 0.25 | μl | 3.25 | μl |
| molecular grade water | 16.75 | μl | 217.75 | μl |
| total volume | 22 | μl | 286 | μl |

5. Pipet 22 μl “IN1” PCR master mix into each well in first row.
6. Pipet 22 μl “IN2” PCR master mix into each well in second row.
7. Pipet 22 μl “IN3” PCR master mix into each well in third row.
8. Pipet 22 μl “IN4” PCR master mix into each well in fourth row.
9. Pipet 22 μl “IN5” PCR master mix into each well in fifth row.
10. Pipet 22 μl “IN6” PCR master mix into each well in sixth row.
11. Add 3 μl cDNA per well according to the following scheme (one sample per column)
12. The positive control in column 11 is the known sample cDNA and the negative control in column 12 is the water control from the cDNA synthesis plate.
13. Cover the plate with a plate seal.
14. Centrifuge the plate 30 sec at 900×g.
15. Place the plate in a thermocycler block and run the program “HIV_INT”
   95° C. 3 min
   40 cycles:
      95° C. 15 sec
      55° C. 20 sec
      72° C. 1 min
   72° C. 8 min
   4° C. forever
16. If not proceeding with the next step immediately, store the plate on ice (for processing the same day) or at −20° C.

The amplicons generated in step 410 may then, in some embodiments, be cleaned up or purified as illustrated in step 413 using either Solid Phase Reversible Immobilization (also referred to as SPR1) or gel cutting methods for size selection known in the related art. For instance, amplicon purification may be performed using the following process:
1. Centrifuge the plate for 30 sec at 900×g.
2. Using an 8-channel multipipettor, pipet 22.5 μl molecular grade water into each well in columns 1-11 of a 96-well, round bottom, PP plate (available from Fisher Scientific).
3. Transfer 22.5 μl PCR product from the PCR plate to each well of the round bottom PP plate; keep the layout the same for the two plates.
4. Add 72 μl SPR1 beads to each well and mix thoroughly by pipetting up and down at least 12 times until the SPR1 bead/PCR mixture is homogeneous.
5. Incubate the plate 10 min at room temperature until supernatant is clear.
6. Place the plate on a 96-well magnetic ring stand (available from Ambion, Inc.) and incubate for 5 min at room temperature.
7. With the plate still on the magnetic ring stand, carefully remove and discard the supernatant without disturbing the beads.
8. Remove the PP plate from the magnetic ring stand and add 200 μl of freshly prepared 70% ethanol.
9. Return the PP plate to the magnetic ring stand. Tap or move the PP plate in a back and forth/circular motion over the magnetic ring stand ˜10 times to agitate the solution and assist in pellet dispersion (the pellet may not fully disperse; this is acceptable).
10. Place the PP plate on the magnetic ring stand and incubate 1 min.
11. With the plate still on the magnetic ring stand, carefully remove and discard the supernatant without disturbing the beads.
12. Repeat steps 8-11. Remove as much of the supernatant as possible.
13. Place the PP plate/magnetic ring stand together on a heat block set at 40° C. until all pellets are completely dry (10-20 min.)
14. Add 10 μl 1×TE (pH 7.6±0.1) to each well. Tap/move the PP plate in the same back and forth/circular motion over the magnetic ring stand until all pellets are dispersed.
15. Place the PP plate on the magnetic ring stand and incubate for 2 min.
16. Pipet the supernatant from each well into a fresh 96-well (yellow) plate. It is difficult to avoid any transfer of pellet in some of the wells; this is acceptable.
17. Cover the plate with a plate seal and store at −20° C.

In the one or more embodiments, it may also be advantageous to quantitate the amplicons. In the present example, amplicon quantitation may be performed using the following process:
1. Using methods known in the art quantify 1 μl of these amplicons with PicoGreen® reagent.
2. Any amplicon quantified at or below 5 ng/μl should be further evaluated on the 2100 Bioanalyzer (available from Agilent Technologies): Load 1 μl of each purified amplicon on a Bioanalyzer DNA chip and run the DNA-1000 series II assay.
   a. If a band of the expected size is present and primer dimers are evident at a molar ratio of 3:1 or less, use the PicoGreen quantification and proceed with amplicon pooling.
   b. If a band of the expected size is present and primer dimers are evident at a molar ratio above 3:1, repeat SPR1 and PicoGreen quantitation, followed by Bioanalyzer analysis to confirm removal of primer dimers.
3. Analyze 1 ul of the negative PCR control reactions on the Bioanalyzer. NO: bands other than primer dimers should be visible

Next, as illustrated in step 415 nucleic acid strands from the amplicons are selected and introduced into emulsion droplets and amplified as described elsewhere in this specification. In some embodiments, two emulsions may be set up per sample, one using an Amplicon A kit and one using an Amplicon B kit both available from 454 Life Sciences Corporation. It will be appreciated that in different embodiments, different numbers of emulsions and/or different kits can be employed. Amplicons may be selected for the final mix using the following process:

In the first embodiment of the method:
1. 6 amplicons for each sample are generated, each of which ideally should be mixed in equimolar amounts for the emPCR reaction. As not all amplicons are generated with equal efficiency and occasionally there is very little amplicon made but a large amount of primer dimers may be present instead. To achieve optimal sequencing results it is important to only use well-quantified and relatively pure (see below) amplicons for the final mix for each sample. Due to the considerable overlap between the various amplicons, not all 6 amplicons are needed for complete coverage of a given sample. Amplicons 1-5 are all of the amplicons required to gain full coverage of this region of interest. Amplicon 6 is to be used interchangeably with amplicon 5 to gain full coverage if amplicon 5 is not generated. When a full set of 5 high quality amplicons is not available follow the rules below for choosing amplicons for the final mix for each sample:
   NB: Amplicon Purity Recommendations
      i. If the amplicon is not recognized as a quantifiable band on the Bioanalyzer, do not use it for the final amplicon mix in 6.2.
      ii. If the molar ratio of primer-dimer to amplicon is 3:1 or more, do not use for the final amplicon mix. This measurement will only be available for the low-concentration amplicons that were further quantified with the Agilent Bioanalyzer assay in 6.1.
      iii. If an amplicon fails the above criteria or is altogether missing, increase the amount of the other overlapping amplicon according to the following scheme:
      iv.
      v. If amplicon IN 1 is missing, double the amount of amplicon IN 2
      vi. If amplicon IN 2 is missing, double the amount of amplicon IN 1
      vii. If amplicon IN 3 is missing, double the amount of amplicon IN 4
      viii. If amplicon IN 4 is missing, double the amount of amplicon IN 3
      ix. If amplicon IN 5 is missing, double the amount of amplicon IN 4
      x.
      xi. If both amplicons IN 1 and IN 2 are missing, the Integrase region cannot be fully sequenced. Repeat PCR for these amplicons if possible.
      xii. If both amplicons IN 3 and IN 4 are missing, there will be a segment of the Integrase region that cannot be fully sequenced (from codon 250-280). If this is acceptable then increase the amount of IN 5 by 150% and increase the amount of IN 1 by 50%. If this is not acceptable, repeat PCR for these amplicons if possible.
      xiii. If both amplicons IN 4 and IN 5 are missing, triple the amount of amplicon IN 3
Also as part of step 415 the following process for mixing and dilution of the amplicons may be employed for use in emPCR:
1. Calculate the concentration in molecules per μl for each of the 6 amplicons derived from a given sample using the following equation:

$$Molecules \text{/} μ 1 = \frac{sample conc [ ng / μ 1 ] * 6.022 * {10}^{23}}{656.6 * {10}^{9} * amplicon length [ bp ]}$$

2. Make a 10⁹ molecules/μl dilution of each of the 6 amplicons:
   To 1 μl of amplicon solution add the following volume of 1×TE:

$$( \frac{molecules \text{/} μ 1 ( from 6.3 .1 )}{{10}^{9}} - 1 ) µ1$$

3. Mix an equal volume of each of the 6 amplicon dilutions, e.g., 10 μl. If either of the amplicons are missing, increase the volumes of overlapping amplicons according to the guidelines in step 405.
4. Make a further dilution of the mixed amplicons to 2×10⁶ molecules/μl by adding 1 μl of the 10⁹ molecules/μl solution to 499 μl 1×TE
5. Store the final dilution (2×10⁶ molecules/0 at −20° C. in a 0.5 ml tube with o-ring cap.

After the amplification the emulsions are broken and beads with amplified populations of immobilized nucleic acids are enriched as illustrated in step 420. For example, DNA-containing beads may be enriched as described elsewhere in this specification, which may include the following process elements:
1. Immediately before setting up emulsions, make a 10-fold dilution of the 2×10⁶ molecules/μl solution from 6.3.4 by adding 10 μl to 90 μl bead wash buffer. Vortex 5 sec. to mix.
2. For each sample, make one A and one B emulsion with 1 cpb (i.e., 12 ul of the above dilution per emulsion (2,400,000 beads)).
3. The two emulsions for a given sample can be pooled during breaking for easier handling.

The enriched beads are then sequenced as illustrated in step 430. In some embodiments, each sample is sequenced as described elsewhere in this specification. For instance, after enrichment and processing for sequencing, load 80,000 beads (incl. the positive control sample) from the combined emulsions per lane on a 70×75 metallized PTP fitted with a 16-lane gasket and sequence on a GS-FLX instrument (available from 454 Life Sciences Corporation).

The GS-FLX sequencing instrument comprises three major assemblies: a fluidics subsystem, a fiber optic slide cartridge/flow chamber, and an imaging subsystem. Reagents inlet lines, a multi-valve manifold, and a peristaltic pump form part of the fluidics subsystem. The individual reagents are connected to the appropriate reagent inlet lines, which allows for reagent delivery into the flow chamber, one reagent at a time, at a pre-programmed flow rate and duration. The fiber optic slide cartridge/flow chamber has a 250 μm space between the slide's etched side and the flow chamber ceiling. The flow chamber also included means for temperature control of the reagents and fiber optic slide, as well as a light-tight housing. The polished (unetched) side of the slide is placed directly in contact with the imaging system.

The cyclical delivery of sequencing reagents into the fiber optic slide wells and washing of the sequencing reaction byproducts from the wells is achieved by a pre-programmed operation of the fluidics system. The program is typically written in a form of an Interface Control Language (ICL) script, specifying the reagent name (Wash, dATPαS, dCTP, dGTP, dTTP, and PPi standard), flow rate and duration of each script step. For example, in one possible embodiment flow rate can be set at 4 mL/min for all reagents with the linear velocity within the flow chamber of approximately ˜1 cm/s. The flow order of the sequencing reagents may be organized into kernels where the first kernel comprises of a PPi flow (21 seconds), followed by 14 seconds of substrate flow, 28 seconds of apyrase wash and 21 seconds of substrate flow. The first PPi flow may be followed by 21 cycles of dNTP flows (dC-substrate-apyrase wash-substrate dA-substrate-apyrase wash-substrate-dG-substrate-apyrase wash-substrate-dT-substrate-apyrase wash-substrate), where each dNTP flow is composed of 4 individual kernels. Each kernel is 84 seconds long (dNTP-21 seconds, substrate flow-14 seconds, apyrase wash-28 seconds, substrate flow-21 seconds); an image is captured after 21 seconds and after 63 seconds. After 21 cycles of dNTP flow, a PPi kernel is introduced, and then followed by another 21 cycles of dNTP flow. The end of the sequencing run is followed by a third PPi kernel. The total run time was 244 minutes. Reagent volumes required to complete this run are as follows: 500 mL of each wash solution, 100 mL of each nucleotide solution. During the run, all reagents were kept at room temperature. The temperature of the flow chamber and flow chamber inlet tubing is controlled at 30° C. and all reagents entering the flow chamber are pre-heated to 30° C.

Subsequently, the output sequence data is analyzed as illustrated in step 440. In some embodiments, SFF files containing flow gram data filtered for high quality are processed using specific amplicon software and the data analyzed.

It will be understood that the steps described above are for the purposes of illustration only and are not intended to be limiting, and further that some or all of the steps may be employed in different embodiments in various combinations. For example, the primers employed in the method described above may be combined with additional primers sets for interrogating other HIV characteristics/regions to provide a more comprehensive diagnostic or therapeutic benefit. In the present example, such combination could be provided “dried down” on a plate and include the described integrase primers as well as some or all of the primers for detection of HIV drug resistance or the tropism region, as well as any other region of interest. Additional examples are disclosed in PCT Application Serial NO: US 2008/003424, titled “System and Method for Detection of HIV Drug Resistant Variants”, filed Mar. 14, 2008; and/or U.S. patent application Ser. No. 12/456,528, titled “System and Method for Detection of HIV Tropism Variants”, filed Jun. 17, 2009, each of which is hereby incorporated by reference herein in its entirety for all purposes.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. A method for detecting low frequency occurrence of one or more HIV sequence variants associated with integrase, comprising the steps of:
(a) generating a cDNA species from a plurality of RNA molecules in an HIV sample population;
(b) amplifying a plurality of first amplicons from the cDNA species, wherein each first amplicon is amplified with a pair of nucleic acid primers capable of amplifying products from clades A, B, C, D, AE and G sub-types;
(c) clonally amplifying the amplified copies of the first amplicons to produce a plurality of second amplicons
(d) determining a nucleic acid sequence composition of the second amplicons;
(e) detecting one or more sequence variants that occur at a frequency of 5% or less in the nucleic acid sequence composition of the second amplicons; and
(f) correlating the detected sequence variants with variation associated with HIV integrase.

2. The method of claim 1, wherein:
the variation associated with HIV integrase is known to be associated with resistance to an integrase inhibitor.

3. The method of claim 1, wherein:
the HIV sample population is derived from a single patient.

4. The method of claim 1, wherein:
the plurality of first amplicons comprises 6 amplicons that provide at least double coverage of an integrase region.

5. The method of claim 4, wherein:
the pair of primers for the plurality of first amplicons comprise a group of primer pairs selected from the group consisting of Int 1F (SEQ ID NO: 1) and Int 1R (SEQ ID NO: 2); Int 2F (SEQ ID NO: 3) and Int 2R (SEQ ID NO: 4); Int 3F (SEQ ID NO: 5) and Int 3R (SEQ ID NO: 6); Int 4F (SEQ ID NO: 7) and Int 4R (SEQ ID NO: 8); Int 5F (SEQ ID NO: 9) and Int 5R (SEQ ID NO: 10); and Int 6F (SEQ ID NO: 11) and Int 6R (SEQ ID NO: 12).

6. The method of claim 1, wherein:
the plurality of first amplicons comprises 4 amplicons that provide single coverage of an integrase region and each amplicon comprising a region of double coverage overlap between neighboring amplicons.

7. The method of claim 6, wherein:
the pair of primers for the plurality of first amplicons comprise a group of primer pairs selected from the group consisting of Int 1F* (SEQ ID NO: 27) and Int 1R* (SEQ ID NO: 28); Int 2F* (SEQ ID NO: 29) and Int 2R* (SEQ ID NO: 30); Int 3F* (SEQ ID NO: 31) and Int 3R* (SEQ ID NO: 32); and Int 4F* (SEQ ID NO: 33) and Int 4R* (SEQ ID NO: 34).

8. The method of claim 1, wherein:
the pair of primers for the first amplicons target conserved regions.

9. The method of claim 1, wherein:
the pair of primers for the first amplicons comprise NO: more than one degenerate position within five positions of a 3′ end of each primer, wherein the degenerate position consists of two nucleotide species possibilities whose combined frequencies add up to >98% frequency.

10. The method of claim 1, wherein:
the pair of primers for the first amplicons target a region in HIV p15 domain and a region in HIV vif domain.

11. The method of claim 1, wherein:
the first amplicon covers a region of HIV associated with HIV integrase functionality.

12. The method of claim 1, wherein:
the second amplicons are amplified using a pair of general primers.

13. The method of claim 1, wherein:
one or more sequence variants are detected at a 99% confidence level.

14. The method of claim 1, wherein:
the one or more sequence variants are detected as a deviation from a consensus sequence.

15. The method of claim 14, wherein:
the consensus sequence is specific to one of the clades.

16. The method of claim 1 wherein:
the nucleic acid composition of the substantially identical copies from at least 400 immobilized populations is determined and one or more of the detected sequence variants occur at a frequency of 1.85% or less.

17. The method of claim 1 wherein:
the nucleic acid composition of the substantially identical copies from at least 10000 immobilized populations is determined and one or more of the detected sequence variants occur at a frequency of 0.74% or less.

18. The method of claim 1 wherein:
the nucleic acid composition of the substantially identical copies from at least 200000 immobilized populations is determined and one or more of the detected sequence variants occur at a frequency of 0.003% or less.

19. The method of claim 1 wherein:
the step of detecting employs an instrument comprising a single detection device capable of detecting signals generated from a plurality of sequencing reactions on a single substrate.

20. The method of claim 1 wherein:
the single substrate comprises a plurality of reaction sites.

21. A kit for detecting one or more HIV sequence variants associated with the integrase region, comprising:
a plurality of the pairs of nucleic acid primers employed to amplify the first amplicons of claim 1.

22. A kit for detecting one or more HIV sequence variants associated with the integrase region, comprising:
one or more pairs of primers selected from the group consisting of Int 1F (SEQ ID NO: 1) and Int 1R (SEQ ID NO: 2); Int 2F (SEQ ID NO: 3) and Int 2R (SEQ ID NO: 4); Int 3F (SEQ ID NO: 5) and Int 3R (SEQ ID NO: 6); Int 4F (SEQ ID NO: 7) and Int 4R (SEQ ID NO: 8); Int 5F (SEQ ID NO: 9) and Int 5R (SEQ ID NO: 10); and Int 6F (SEQ ID NO: 11) and Int 6R (SEQ ID NO: 12).

23. A kit for detecting one or more HIV sequence variants associated with the integrase region, comprising:
one or more pairs of primers selected from the group consisting of Int 1F* (SEQ ID NO: 27) and Int 1R* (SEQ ID NO: 28); Int 2F* (SEQ ID NO: 29) and Int 2R* (SEQ ID NO: 30); Int 3F* (SEQ ID NO: 31) and Int 3R* (SEQ ID NO: 32); and Int 4F* (SEQ ID NO: 33) and Int 4R* (SEQ ID NO: 34).
